(19) United States
(12) Patent Application Publication
(10) Pub. No.: US 2016/0068884 A1
(43) Pub. Date: Mar. 10, 2016

(54) METHODS FOR DETERMINING PRESENCE OR ABSENCE OF GLYCAN EPITOPES ON GLYCOPROTEINS

(71) Applicant: Bio-Techne Corporation, Minneapolis, MN (US)

(72) Inventors: Zhengliang L. Wu, Edina, MN (US); Alexander Kalyuzhny, Roseville, MN (US)

(21) Appl. No.: 14/847,564

(22) Filed: Sep. 8, 2015

Related U.S. Application Data
(60) Provisional application No. 62/047,904, filed on Sep. 9, 2014.

(51) Int. Cl. C12Q 1/48; G01N 33/53

(52) U.S. Cl. CPC C12Q 1/48; G01N 33/5308; G01N 2333/91091; G01N 2333/924; G01N 2400/02; G01N 2400/00

(57) ABSTRACT

The disclosure relates to in vitro methods of detecting presence or absence of a target carbohydrate on a glycoprotein. The disclosure also relates to in vitro methods of detecting presence or absence of a glycan epitope on a glycoprotein.

### RELATED APPLICATIONS

This application claims the benefit of U.S. Provisional Patent Application No. 62/047,904, filed Sep. 9, 2014, the entire contents of which are incorporated herein by reference. This application also relates to U.S. utility application titled “Molecular Labeling Methods” and filed concurrently herewith under attorney document number 10805.125.3, the entire contents of which are incorporated herein by reference.

### BACKGROUND

A majority of expressed mammalian proteins are glycoproteins. Glycan epitopes are commonly found on glycoproteins with various biological functions. However, glycan epitopes are difficult to probe and analyze due a lack of antibodies specific to those epitopes. There are many glycan epitopes of interest. For example, Lewis X, and its sialylated version, sialyl Lewis X structure, are epitopes commonly found in circulations and play key roles in leukocyte honing. Also, T and Tn antigens are O-linked glycan epitopes commonly found on cancer cells. As such, it would be desirable to provide a method of determining presence or absence of glycan epitopes on glycoproteins.

### SUMMARY

Improved methods and systems for determining presence of absence of glycan epitopes on target glycoproteins are described. Glycosylation is an enzymatic process that attaches glycans to molecules. For example, proteins often form conjugates in the form of glycoproteins or proteoglycans through glycosylation. The current methods involve removing a target carbohydrate to generate a glycan acceptor site through a glycosidase treatment and incorporating a replacement carbohydrate into the site using a glycosyltransferase. The methods further involve detecting the replacement carbohydrate via click chemistry to identify the presence or absence the target carbohydrate. The presence or absence of the target carbohydrate can then be correlated to the presence or absence of a glycan epitope.

Some embodiments provide an in vitro method of determining presence or absence of a target carbohydrate on a target glycoprotein, comprising: (a) providing a sample containing a target glycoprotein, (b) treating the sample with glycosidase, wherein the glycosidase removes a target carbohydrate if present on the target glycoprotein, thereby creating a vacant glycosylation acceptor site, (c) treating the sample with a glycosyltransferase to incorporate a replacement carbohydrate at the vacant glycosylation acceptor site if present, wherein the replacement carbohydrate includes a click chemistry moiety, (d) adding a label to the sample, wherein the label includes a click chemistry moiety that reacts to the click chemistry moiety of the replacement carbohydrate such that that the label attaches to the replacement carbohydrate, (e) separating the sample using a separation method (e.g., an electrophoresis method), (f) determining the presence or absence of label attached to the replacement carbohydrate in the separated sample (e.g., with a blotting method), and (g) correlating the presence of the label to the presence of the target carbohydrate or correlating the absence of the label to the absence of the target carbohydrate. The method can also include correlating the presence of the target carbohydrate to the presence of a glycan epitope or correlating absence of the target carbohydrate to absence of the glycan epitope.

In other embodiments, an in vitro method of determining presence or absence of a target carbohydrate on a target glycoprotein includes (a) providing a sample containing a target glycoprotein, (b) aliquoting the sample into a first aliquot and a second aliquot, (c) treating the first aliquot with a glycosidase and leaving the second aliquot untreated, wherein the glycosidase removes a target carbohydrate if present to create a vacant glycosylation acceptor site on the target glycoprotein, (d) treating each the first aliquot and the second aliquot with a glycosyltransferase, wherein the glycosyltransferase incorporates a replacement carbohydrate into the vacant glycosylation acceptor site if present, wherein the replacement carbohydrate includes a click chemistry moiety, (e) adding a label to each the first aliquot and the second aliquot, wherein the label includes a click chemistry moiety that reacts to the click chemistry moiety of the replacement carbohydrate such that that the label attaches to the replacement carbohydrate, (f) performing a separation method (e.g., an electrophoresis method) on each the first aliquot and the second aliquot, (g) determining presence or absence of label attached to the replacement carbohydrate in each the first aliquot and the second aliquot (e.g., with a blotting method), and (h) correlating presence or absence of label in the first aliquot and the second aliquot to presence of a target carbohydrate. The method can also include correlating the presence of the target carbohydrate to the presence of a glycan epitope or correlating absence of the target carbohydrate to absence of the glycan epitope. As used herein, the term “aliquot” merely means a part of the whole and each of the recited aliquots can be uneven divisions of the whole or even divisions of the whole.

Other embodiments provide an in vitro method of determining presence or absence of target carbohydrates, comprising: (a) providing a sample containing a target glycoprotein; (b) aliquoting the sample into a first aliquot and a second aliquot, (c) treating the first aliquot with a glycosidase and leaving the second aliquot untreated, wherein the glycosidase in the removes a target carbohydrate if present to create a vacant glycosylation acceptor site on the target glycoprotein, (d) further aliquoting the first aliquot into a first subgroup of aliquots and the second aliquot into a second subgroup of aliquots, (e) treating each aliquot in the first subgroup and the second subgroup with a different glycosyltransferase, wherein each glycosyltransferase includes a single glycosyltransferase or a plurality of glycosyltransferases, wherein each glycosyltransferase incorporates a target carbohydrate into the vacant glycosylation acceptor site if present, wherein the target carbohydrate includes a click chemistry moiety, (f) adding a label to each aliquot in the first subgroup and the second subgroup, wherein the label includes a click chemistry moiety that reacts to the click chemistry moiety of the target carbohydrate such that that the label attaches to the target carbohydrate, (g) performing a separation method (e.g., an electrophoresis method) on each aliquot in the first subgroup and the second subgroup, (h) determining presence or absence of the label in each aliquot in the first subgroup and the second subgroup (e.g., with a blotting method), and (i) correlating presence or absence of label to presence or absence of target carbohydrates. The method can further include correlating presence of a target carbohydrate in the target carbohydrates to presence of a glycan epitope. In some cases, the glycosyltransferases includes at least a first glycosyltransferase and a second glycosyltransferase, wherein each glycosyltransferase is different.

Other embodiments provide an in vitro method of determining presence or absence of target carbohydrates, comprising: (a) providing a sample containing a target glycoprotein; (b) aliquoting the sample into a plurality of aliquots, (c) leaving one of the aliquots in the plurality of aliquots untreated and treating each of the remaining aliquots with a different glycosidase, wherein each glycosidase includes a single glycosidase or a plurality of glycosidases, wherein each glycosidase removes a target carbohydrate if present to create a vacant glycosylation acceptor site on the target glycoprotein, (d) further aliquoting each aliquot in the plurality of aliquots into a subgroup of aliquots, (e) treating each aliquot in each subgroup with a different glycosyltransferase, wherein each glycosyltransferase includes a single glycosyltransferase or a plurality of glycosyltransferases, wherein each glycosyltransferase incorporates a target carbohydrate into the vacant glycosylation acceptor site if present, wherein the target carbohydrate includes a click chemistry moiety, (f) adding a label to each aliquot in each subgroup, wherein the label includes a click chemistry moiety that reacts to the click chemistry moiety of a target carbohydrate such that that the label attaches to the target carbohydrate, (g) performing a separation method (e.g., an electrophoresis method) on each aliquot in each subgroup, (h) determining presence or absence of the label attached to target carbohydrates in each aliquot in each subgroup (e.g., with a blotting method), and (i) correlating presence or absence of the label to presence or absence of target carbohydrates. The method can further include correlating presence of a target carbohydrate in the target carbohydrates to presence of a glycan epitope. In some cases, the glycosidases includes at least a first glycosidase and a second glycosidase, wherein each glycosidase is different. Likewise, in some cases, the glycosyltransferases includes at least a first glycosyltransferase and a second glycosyltransferase, wherein each glycosyltransferase is different.

In each of the methods, the replacement carbohydrate can include a click chemistry moiety selected from one of an azido group or an alkyne group and the label can include a click chemistry moiety selected from the other of the azido group or the alkyne group. Also, the label can include a label selected from a biotin molecule, a fluorescent molecule or a luminescent molecule. Also, the target carbohydrate(s) can be selected from the group consisting of sialic acid, fucose, GlcNAc, GalNAc, galactose, mannose and xylose and combinations thereof. Likewise, the glycosidase(s) can be selected from the group consisting sialidase, fucosidase, hexosaminidase and galactosidase or combinations thereof. Also, the glycosyltransferase(s) can be selected from the group consisting of sialyltransferases, fucosyltransferases, GlcNAc transferases, GalNAc transferases, galactosyltransferases, glucosyltransferases, xylosyltransferases, mannosyltransferases and combinations thereof.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a flow diagram showing a method of determining the presence or absence of a target carbohydrate on a target molecule according to various embodiments.

FIG. 2 is a flow diagram showing a method of determining the presence or absence of a sialic acid on a target glycoprotein according to various embodiments.

FIG. 3 is a flow diagram showing a method of determining the presence or absence of a target carbohydrate on a target glycoprotein according to various embodiments.

FIG. 4 is a flow diagram showing a method of determining the presence or absence of target carbohydrates on a target glycoprotein according to various embodiments.

FIG. 5 is a flow diagram showing a method of determining the presence or absence of target carbohydrates on a target glycoprotein according to various embodiments.

FIG. 6 is a table showing nine sialyltransferases used for probing fetal bovine fetuin according to various embodiments.

FIG. 7 shows images of the results of probing fetal bovine fetuin according to various embodiments.

FIG. 8 shows additional images of the results of probing fetal bovine fetuin according to various embodiments.

FIG. 9 shows additional images of the results of probing fetal bovine fetuin according to various embodiments.

FIG. 10 shows additional images of the results of probing fetal bovine fetuin according to various embodiments.

FIG. 11 shows additional images of the results of probing fetal bovine fetuin according to various embodiments.

FIG. 12 shows an illustration of the flow diagram shown in FIG. 3.

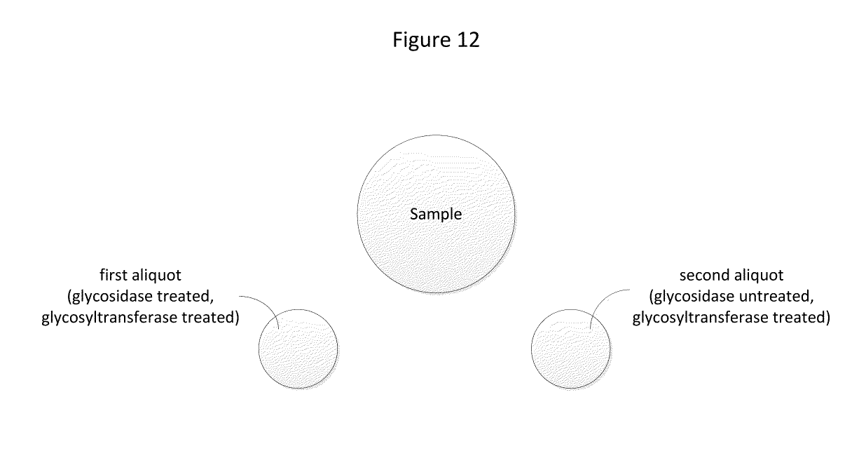

FIG. 13 shows an illustration of the flow diagram shown in FIG. 4.

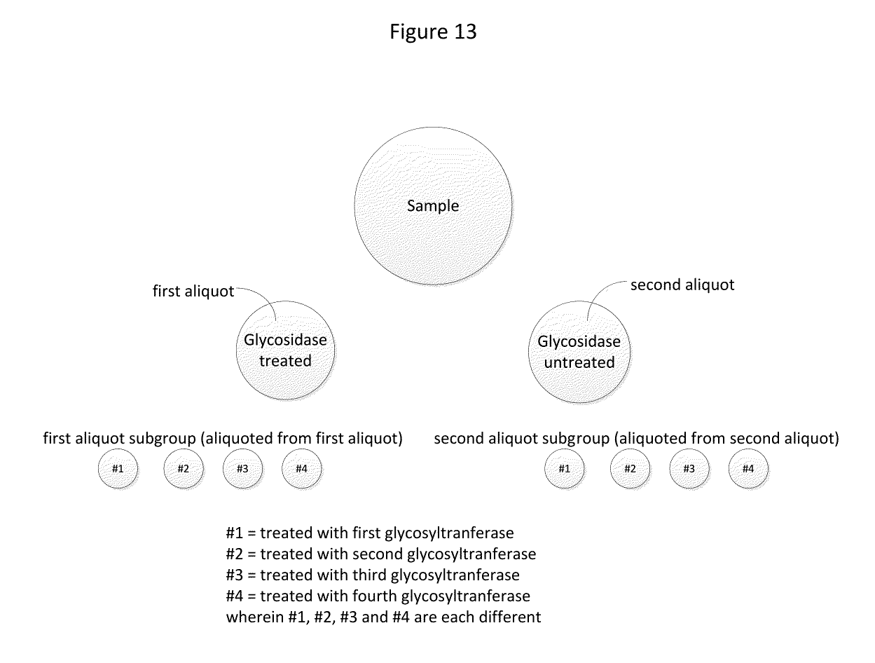

FIG. 14 shows an illustration of the flow diagram shown in FIG. 5.

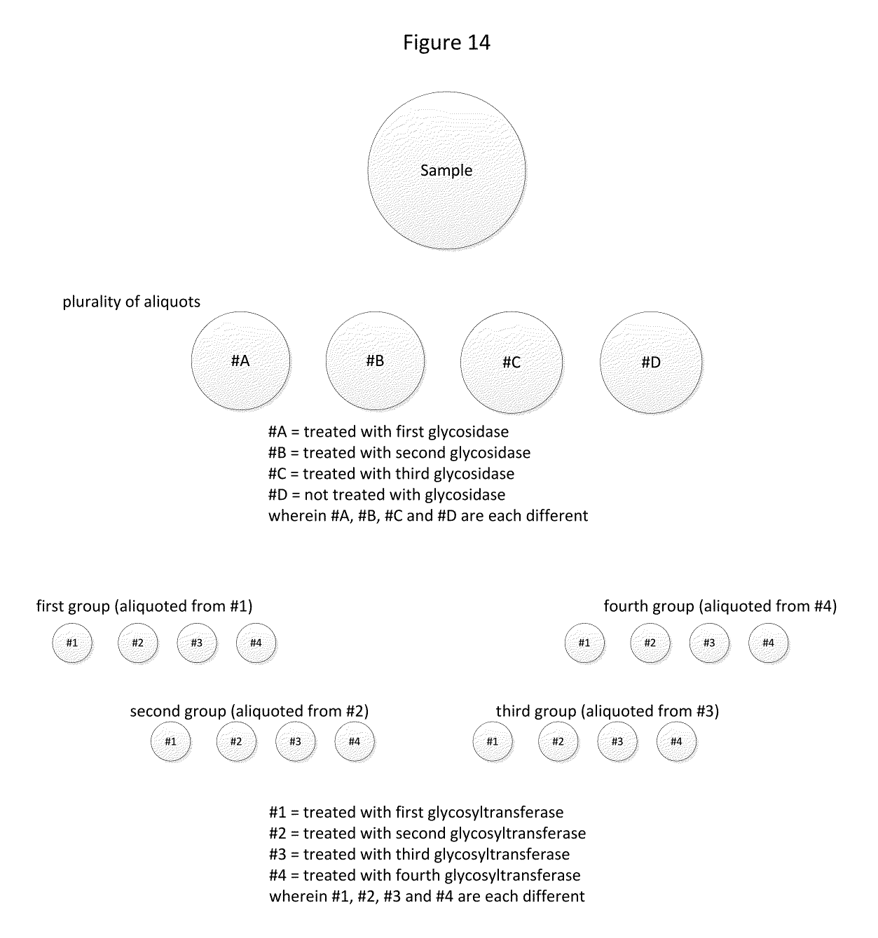

### DETAILED DESCRIPTION

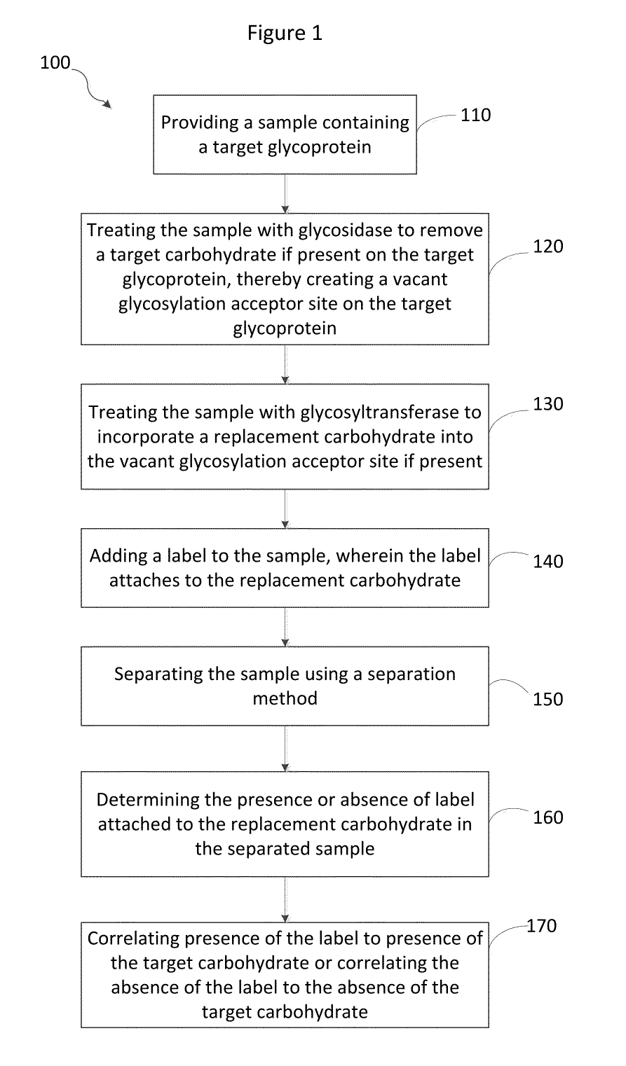

Some embodiments provide in vitro methods of determining presence or absence of a target carbohydrate on a target glycoprotein. One exemplary in vitro method 100 is represented through the flow chart shown in FIG. 1. The method 100 generally includes a step 110 of providing a sample containing a target glycoprotein, a step 120 of treating the sample with glycosidase, wherein the glycosidase removes a target carbohydrate, if present, on the target glycoprotein, thereby creating a vacant glycosylation acceptor site on the target glycoprotein, a step 130 of treating the sample with a glycosyltransferase to incorporate a replacement carbohydrate into the vacant glycosylation acceptor site, if present, a step 140 of adding a label to the sample, wherein the label attaches to the replacement carbohydrate, a step 150 of separating the sample using a separation method, a step 160 of determining presence or absence of label attached to the replacement carbohydrate in the separated sample, and a step 170 of correlating presence of the label to the presence of the target carbohydrate or correlating the absence of the attached clickable label to the absence of the carbohydrate. Each of these steps will now be discussed in more detail.

The method 100 includes a step 110 of providing a sample containing a target glycoprotein. The target glycoprotein can be provided in a solution, for example a buffer solution. Suitable buffer solutions include Tris, HEPES, MES or any other kind of Good's buffer solutions.

The method also includes a step 120 of treating the sample with a glycosidase, wherein the glycosidase removes a target carbohydrate if present on the target glycoprotein, thereby creating a vacant glycosylation acceptor site. Possible target carbohydrates that can be removed by a glycosidase include sialic acid, fucose, GlcNAc, GalNAc, galactose, mannose and xylose. Possible glycosidase that can be used include sialidase, fucosidase, hexosaminidase and galactosidase. In some cases, the glycosidase is a sialidase and the carbohydrate is removed by sialidase treatment.

In some cases, the treating the sample with glycosidase comprises mixing the sample with a buffer containing glycosidase for a period of time and at a temperature and then removing or inactivating glycosidase after the period of time. In certain cases, the removing the glycosidase comprises separating the glycosidase from the sample on a chromatograph column. In other cases, the inactivating the glycosidase comprises heating the sample to a temperature for a period of time. In some cases, the temperature is a temperature in the range of between 55° C. and 98° C. Also, in some cases, the period of time is a period in the range of between 2 to 10 minutes.

In some embodiments, the target carbohydrate that is removed is a terminal monosaccharide, such as a terminal sialic acid. In other embodiments, the target carbohydrate that is removed is an oligosaccharide, such as an O-glycan. In some cases, an O-glycan is removed and is one selected from Core-1, Core-2, Core-3, Core-4, Core-5, Core-6, Core-7 and Core-8 O-glycans. Once the target carbohydrate residue is removed, the target protein has a vacant glycosylation acceptor site where the target carbohydrate used to be.

The glycosidase a specific glycosidase or a combination of specific glycosidases that specifically removes the target carbohydrate. In some cases, the target carbohydrate is a monosaccharide and is removed by a single glycosidase. In other cases, the target carbohydrate is an oligosaccharide and is removed by a combination of glycosidases. For example, Thomsene-Friedenreich antigen and its sialyl version (sialyl-T antigen) are two O-glycans known to be present on bovine serum fetuin that can be removed with the endoglycosidase E. faecalis Endo-EF and recombinant C. perfringens neuraminidase. Other various O-glycans can be removed with a combination of a galactosidase or an endoglycosidase E. faecalis Endo-EF and C. perfringens neuraminidase.

The method also includes a step 130 of treating the sample with a glycosyltransferase to incorporate a replacement carbohydrate into the vacant glycosylation acceptor site if the site is present. The replacement carbohydrate includes a click chemistry moiety that can be used in a click chemistry reaction, such as an azido or an alkyne group. In some cases, the replacement carbohydrate is a monosaccharide. The glycosyltransferases used to incorporate replacement carbohydrates into the vacant glycosylation acceptor sites can include but are not limited to sialyltransferases, fucotransferases, GkNAc transferases, GalNAc transferases, galactosyltransferases, glucosyltransferases, xylosyltransferases and mannosyltransferases.

The method further includes a step 140 of adding a label to the sample so that a click chemistry reaction is performed. Click chemistry is a way to quickly and reliably join small units together. It is not a single specific reaction, but refers to a general way of joining small modular units. The label includes a click chemistry moiety that reacts to the click chemistry moiety of the replacement carbohydrate such that the label attaches to the replacement carbohydrate. In some cases, the replacement carbohydrate includes an azido group and the label includes an alkyne group. In other cases, the replacement carbohydrate includes an alkyne group and the label includes an azido group. The clickable label can be a reporter molecule such as a colorimetric label, a biotin label, a luminescent label or a fluorescent label.

In the method 100, a high level of specificity can be achieved by two levels of enzymatic reaction. The first level is by the selection of a glycosidase that removes a target carbohydrate to create a vacant glycosylation acceptor site. The second level is by the selection of a glycosyltransferase that only recognizes the specific vacant glycosylation acceptor site. Also, the glycosidase has a substrate specificity that matches or overlaps with a substrate specificity of the glycosyltransferase. In some cases, the glycosidase is a sialidase and the glycosyltransferase is a sialyltransferase, wherein the substrate specificity of the sialidase matches or overlaps with that of the sialyltransferase. For example, a sialidase specific for 6-O sialic acid may be used in pair with a 6-O specific sialyltransferase. Following are exemplary glycosidase-glycosyltransferase pairs that can be used to replace certain monosaccharides:

TABLE

| Monosaccharides | | |
|---|---|---|
| that are replaced | Glycosidases | Glycosyltransferases |
| Sialic acid | α2-3 specific sialidases | STGal1, 2, 3, 4, 5, |
| | α2-6 specific sialidases | ST6Gal1, 2, |
| | α2-8 specific sialidases | ST6GalNAc1, 2, 3, 4, 5, 6 |
| | | ST8Sia1, 2, 3, 4. |
| fucose | α-fucosidase | FUT1, 2, 3, 4, 5, |
| | | 6, 7, 8, 9, 10, 11 |
| GlcNAc | α-hexosaminidases | MGAT1, 2, 3, 4, 5 |
| | β-hexosaminidases | GCNT1, 2, 3, 4, 5, 6 |
| | | B3GNT1, 2, 3, 4, 5, 6, 7 |
| | | A4GNT |
| GalNAc | α-hexosaminidases | GALNT1, 2, 3 . . . to 20 |
| | β-hexosaminidases | |
| T antigen | O-glycosidase (Endo EF) | GALNT1, 2, 3 . . . to 20 |

The method further includes a step 150 of separating the sample using a separation method such as electrophoresis and a step 160 of determining presence or absence of label attached to the replacement carbohydrate in the separated sample. In some cases, step 160 is performed by using visualization method such as a chemiluminescent method or a blotting method such as Western blotting.

The method further includes a step 160 of correlating the presence of the label to the presence of the target carbohydrate or correlating the absence of the label to the absence of the target carbohydrate. In some cases, the method includes further correlating the presence of the target carbohydrate to the presence of a specific glycan epitope or correlating the absence of the target carbohydrate to the absence of a specific glycan epitope.

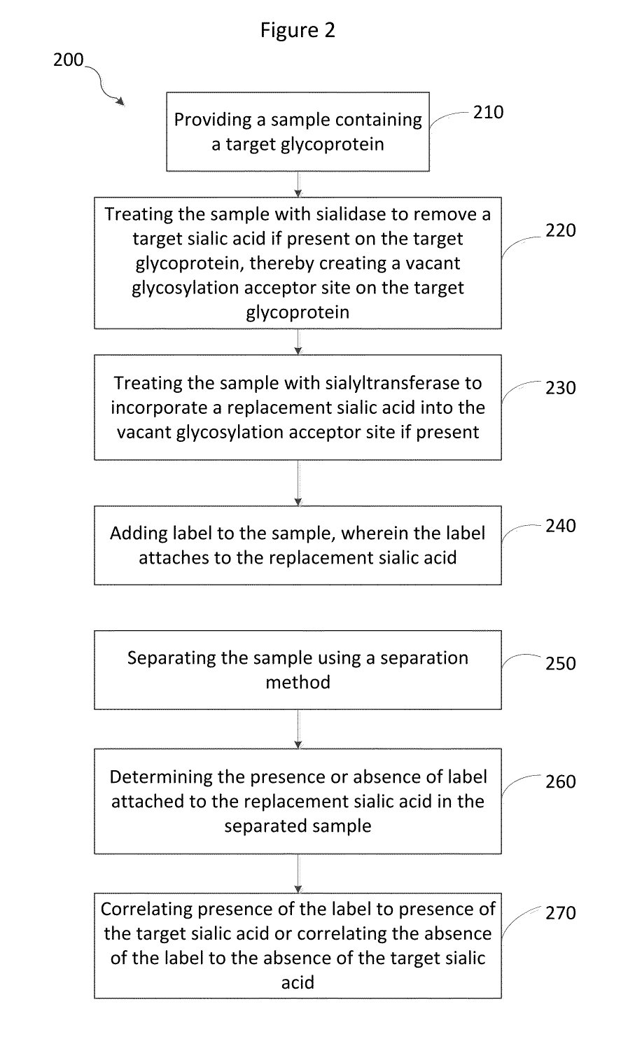

Some embodiments provide in vitro methods of determining the presence or absence of a target sialic acid on a target glycoprotein. For example, one exemplary in vitro method 200 is represented through the flow chart shown in FIG. 2. The method 200 includes a step 210 of providing a sample containing a target glycoprotein, a step 220 of treating the sample with sialidase, wherein the sialidase removes a target sialic acid if present on the target glycoprotein, thereby creating a vacant glycosylation acceptor site on the target glycoprotein, a step 230 of treating the sample with sialyltransferase to incorporate a replacement sialic acid onto the vacant glycosylation acceptor site, if present, a step 240 of adding a label to the sample, wherein the label attaches to the replacement sialic acid, if present, a step 250 of separating the sample using a separation method, a step 260 of determining presence or absence of label attached to the replacement sialic acid in the separated sample, and a step 270 of correlating the presence of the label to the presence of the target sialic acid or correlating the absence of the label to the absence of the target sialic acid. Steps 210 through 260 can be performed as already described for steps 110-160.

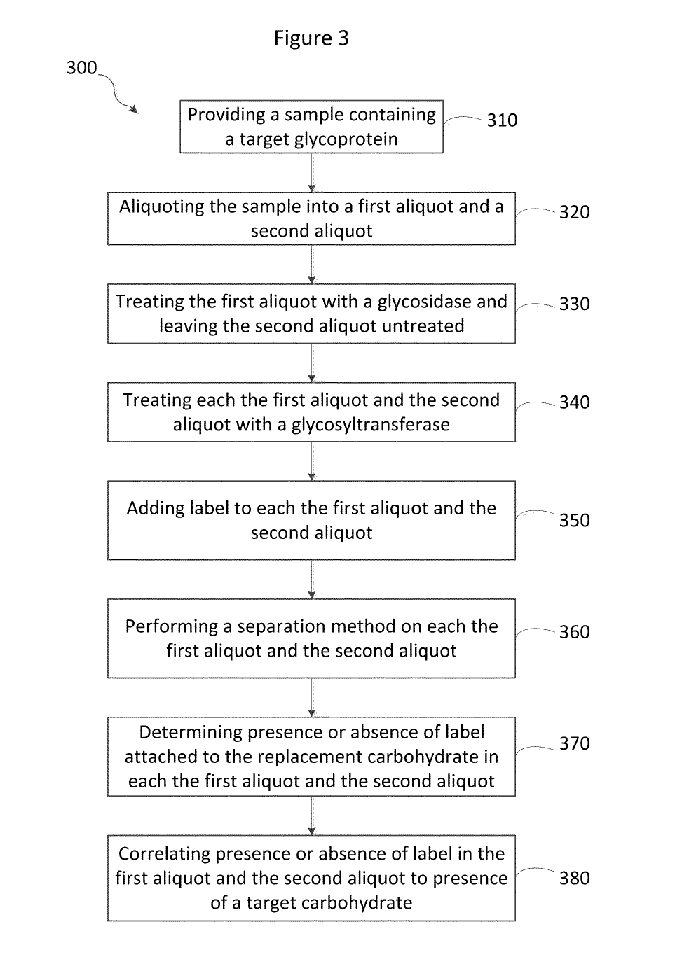

Another in vitro method 300 of determining presence or absence of a target carbohydrate on a target glycoprotein is represented through the flow chart shown in FIG. 3. The method 300 generally includes a step 310 of providing a sample containing a target glycoprotein, a step 320 of aliquoting the sample into a first aliquot and a second aliquot, a step 330 of treating the first aliquot with a glycosidase and leaving the second aliquot untreated, wherein the glycosidase removes a target carbohydrate if present to create a vacant glycosylation acceptor site on the target glycoprotein, a step 340 of treating each the first aliquot and the second aliquot with a glycosyltransferase, wherein the glycosyltransferase incorporates a replacement carbohydrate into the vacant glycosylation acceptor site if present, wherein the replacement carbohydrate includes a click chemistry moiety, a step 350 of adding a label to each the first aliquot and the second aliquot, wherein the label includes a click chemistry moiety that reacts to the click chemistry moiety of the replacement carbohydrate such that that the label attaches to the replacement carbohydrate, a step 360 performing a separation method (such as electrophoresis) on each the first aliquot and the second aliquot, a step 370 of determining presence or absence of label attached to the replacement carbohydrate (such as a blotting method) in each the first aliquot and the second aliquot, and a step 380 of correlating presence or absence of label to presence of a target carbohydrate. The method can also include correlating the presence of the target carbohydrate to the presence of a glycan epitope or correlating absence of the target carbohydrate to absence of the glycan epitope. Again, as used herein, the term “aliquot” merely means a part of the whole and each of the recited aliquots can be uneven divisions of the whole or even divisions of the whole.

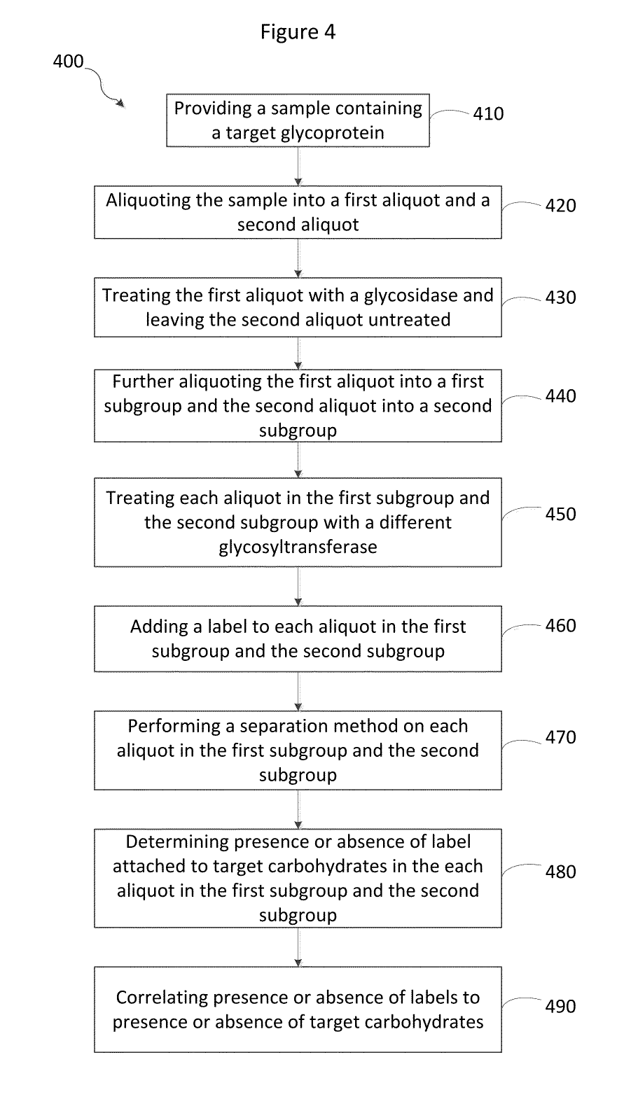

Another method 400 of determining presence or absence of a target carbohydrate on a target glycoprotein is represented through the flow chart shown in FIG. 4. The method 400 generally includes a step 410 of providing a sample containing a target glycoprotein, a step 420 of aliquoting the sample into a first aliquot and a second aliquot, a step 430 of treating the first aliquot with a glycosidase and leaving the second aliquot untreated, wherein the glycosidase removes a target carbohydrate if present to create a vacant glycosylation acceptor site on the target glycoprotein, a step 440 of further aliquoting the first aliquot into a first subgroup of aliquots and the second aliquot into a second subgroup of aliquots, a step 450 of treating each aliquot in the first subgroup and the second subgroup with a different glycosyltransferase, wherein each glycosyltransferase includes a single glycosyltransferase or a plurality of glycosyltransferases, wherein each glycosyltransferase incorporates a target carbohydrate into the vacant glycosylation acceptor site if present, wherein the target carbohydrate includes a click chemistry moiety, a step 460 of adding a label to each aliquot in the first subgroup and the second subgroup, wherein the label includes a click chemistry moiety that reacts to the click chemistry moiety of the target carbohydrate such that that the label attaches to the target carbohydrate, a step 470 of performing a separation method (e.g., an electrophoresis method) on each aliquot in the first subgroup and the second subgroup, a step 480 of determining presence or absence of the label in each aliquot in the first subgroup and the second subgroup (e.g., with a blotting method), and a step 490 of correlating presence or absence of label to presence or absence of target carbohydrates. The method can further include correlating presence of a target carbohydrate in the target carbohydrates to presence of a glycan epitope. In some cases, the glycosyltransferases includes at least a first glycosyltransferase and a second glycosyltransferase, wherein each glycosyltransferase is different.

Also, the first subgroup and the second subgroup can each be treated with the same panel of glycosyltransferases. For example, in some cases, each the first subgroup and the second subgroup is treated with a panel of #1, #2 . . . or #1, #2, #3 . . . or #1, #2, #3, #4 . . . or #1, #2, #3, #4, #5 . . . wherein each of #1, #2, #3, #4, and #5 includes different glycosyltransferases.

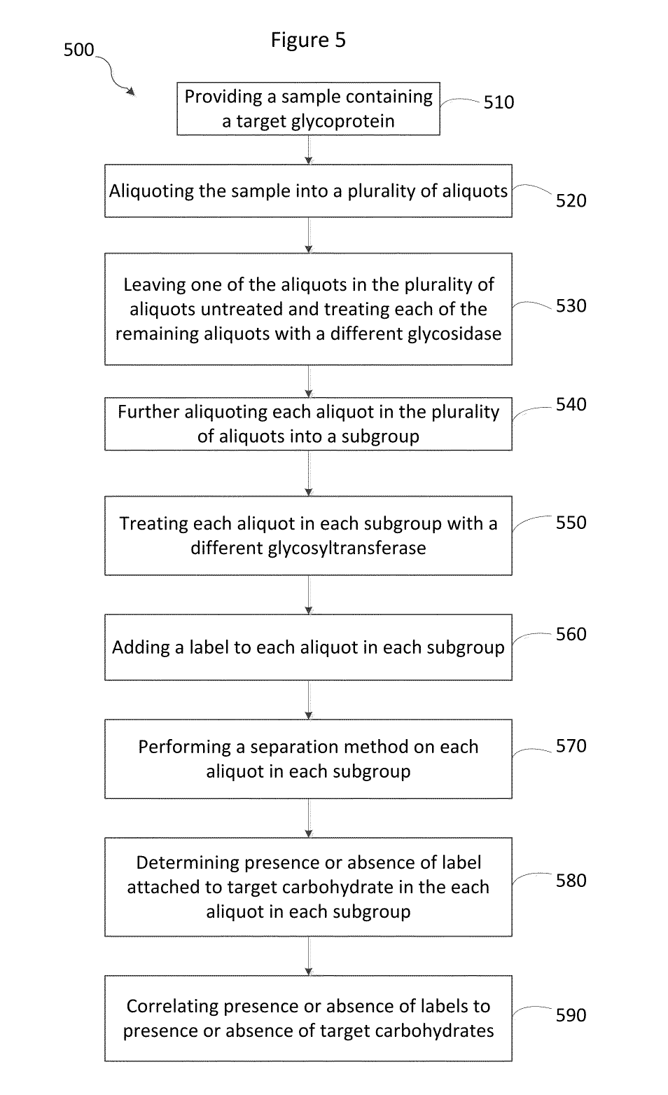

Another method 500 of determining presence or absence of a target carbohydrate on a target glycoprotein is represented through the flow chart shown in FIG. 5. The method 500 generally includes a step 510 of providing a sample containing a target glycoprotein, a step 520 of aliquoting the sample into a plurality of aliquots, a step 530 of leaving one of the aliquots in the plurality of aliquots untreated and treating each of the remaining aliquots with a different glycosidase, wherein each glycosidase includes a single glycosidase or a plurality of glycosidases, wherein each glycosidase removes a target carbohydrate if present to create a vacant glycosylation acceptor site on the target glycoprotein, a step 540 of further aliquoting each aliquot in the plurality of aliquots into a subgroup of aliquots, a step 550 of treating each aliquot in each subgroup with a different glycosyltransferase, wherein each glycosyltransferase includes a single glycosyltransferase or a plurality of glycosyltransferases, wherein each glycosyltransferase incorporates a target carbohydrate into the vacant glycosylation acceptor site if present, wherein the target carbohydrate includes a click chemistry moiety, a step 560 of adding a label to each aliquot in each subgroup, wherein the label includes a click chemistry moiety that reacts to the click chemistry moiety of a target carbohydrate such that that the label attaches to the target carbohydrate, a step 570 of performing a separation method (e.g., an electrophoresis method) on each aliquot in each subgroup, a step 580 of determining presence or absence of the label attached to target carbohydrates in each aliquot in each subgroup (e.g., with a blotting method), and a step 590 of correlating presence or absence of the label to presence or absence of target carbohydrates. The method can further include correlating presence of a target carbohydrate in the target carbohydrates to presence of a glycan epitope.

In some cases, the glycosidases include at least a first glycosidase and a second glycosidase, wherein each glycosidase is different. Likewise, in some cases, the glycosyltransferases includes at least a first glycosyltransferase and a second glycosyltransferase, wherein each glycosyltransferase is different. Also, each subgroup can each be treated with the same panel of glycosyltransferases. For example, in some cases, each subgroup is treated with a panel of #1, #2 . . . or #1, #2, #3 . . . or #1, #2, #3, #4 . . . or #1, #2, #3, #4, #5 . . . wherein each of #1, #2, #3, #4, #5 includes a different glycosyltransferase.

### EXPERIMENTAL

### Materials

Fetal bovine fetuin and asialo-fetuin were obtained from Sigma Aldrich. Recombinant human enzymes ST3Gal1, ST3Gal2, ST6Gal1, ST6Gal2, ST6GalNAc2, ST6GalNAc4, ST6GalNAc6, ST8Sia1, ST8Sia4, recombinant C. perfringens neuraminidase, streptavidin conjugated horseradish peroxidase (strep-HRP) and its ECL substrate were obtained from Bio-Techne/R&D Systems. CMP-azido sialic acid and a biotin alkyne adduct D were synthesized in house. Desialylated fetuin (d-fetuin) was prepared from fetal bovine fetuin with C. perfringens neuraminidase treatment. Both fetuin and d-fetuin were further purified with gel filtration before use.

CMP-azido-sialic acid was synthesized as follows. 2 μmol CTP plus 2 μmol 9-azido sialic acid were mixed with 20 μg of recombinant N. meningitidis Sia B and 5 μg of recombinant yeast pyrophosphatase S. cerevisiae PPA1 in 0.5 mL of buffer of 25 mM Tris and 10 mM MgCl₂ at pH 7.5. The mixture was incubated at 37° C. for 1 hour to form CMP-azido-sialic acid.

UDP-azido-GalNAc was synthesized as follows. Uridine 5′-(trihydrogen diphosphate), 2′-deoxy-, P′-[2-[(2-azidoacetyl)amino]-2-deoxy-α-D-galactopyranosyl]ester ammonium salt was synthesized. Glucosamine 1-phospate was treated with NHS-2-azidoacetate and the crude intermediate, after conversion to the triethylammonium salt, was reacted directly with uridine 5′-monophosphomorpholidate 4-morpholine-N,N-dicyclohexylcarboxamidine salt. Purification using HILIC preparative chromatography and lyophilization gave uridine 5′-(trihydrogen diphosphate), 2′-deoxy-, P′-[2-[(2-azidoacetyl)amino]-2-deoxy-α-D-galactopyranosyl]ester ammonium salt.

Biotin alkyne adduct D 3-(2′-(2″-(2″-Amide-D-biotin-ethoxy)ethoxy)ethoxy) prop-1-yne was synthesized as follows. 2-(2-(2-(prop-2-yn-1-yloxy)ethoxy)ethoxy)ethanamine was synthesized and converted into Biotin alkyne adduct D 3-(2′-(2″-(2′″-Amide-D-biotin-ethoxy)ethoxy)ethoxy) prop-1-yne via reaction with biotin.

### Methods

### Glycosidase Treatment

A fetuin sample was mixed with C. perfringens neuraminidase or PNGase F at the mass ratio of 100:1 in a buffer of 25 mM Tris, 150 mM NaCl at pH 7.5, and at room temperature for 20 minutes. The neuraminidase treated sample was further purified on a Superdex 200 gel filtration column (GE Healthcare).

### Glycosyltransferase Probing Reaction

Sialyltransferase probing reactions were carried out as following: A sample of 10 μg fetuin or d-fetuin was mixed with 0.3 nmol of CMP-azido-Sialic acid, 2 μg of sialyltransferase in 50 μL of 25 mM Tris, supplemented with 10 mM of MnCl₂ and 150 mM NaCl at pH 7.5, and incubated at 37° C. for a minimum of 20 minutes. For the GlcNAc transferase probing reaction, 10 μg glycoprotein sample was mixed with 0.3 nmol of UDP-azido-GalNAc, 2 μg of GlcNAc transferase, in 50 μL of 25 mM Tris supplemented with 10 mM of MnCl₂ and 150 mM NaCl at pH 7.5 and incubated at 37° C. for minimum of 20 minutes.

### Click Chemistry Reaction

For the click chemistry reactions, ascorbic acid, CuCl₂ and biotin alkyne adduct D were directly added to the glycosyltransferase reaction at a final concentration of 2 mM, 0.1 mM and 0.1 mM, respectively. The mixture was incubated at room temperature for a minimum of 30 minutes.

### SDS-PAGE and Gel Blotting

Once the click chemistry reaction was complete, the samples were separated on 12% SDS-PAGE gels. The gels were visualized with UV in the presence of trichlorethanol (TCE staining), which reacts with the indole ring of the amino acid tryptophan[48]. Next, the gels were blotted to nitrocellulose paper under 25 volts for 30 minutes. The blots were blocked with 10% fat-free milk for 10 minutes and washed thoroughly with TBST buffer containing 25 mM Tris, pH 7.6, 137 mM NaCl and 0.01% Tween. The blots were then probed with strep-HRP at 30 ng/mL for 30 minutes and washed three times with TBST buffer for a total of 30 minutes. The blots were finally visualized with enhanced chemiluminescence (ECL) peroxidase substrate in the same way as Western blotting.

### Results

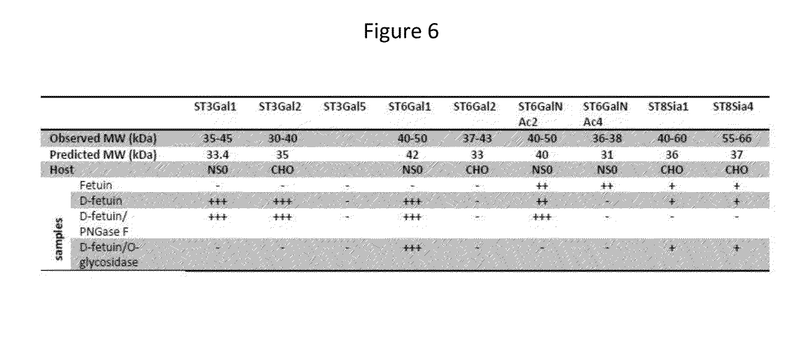

Fetal bovine fetuin was first labeled with a panel of nine recombinant sialyltransferases (ST3Gal1, ST3Gal2, ST3Gal5, ST6Gal1, ST6Gal2, ST6GalNAc2, ST6GalNAc4, and ST8Sia1 and ST8Sia4) without or with prior treatment with certain glycosidases under native conditions. The nine sialyltransferases used for the labeling on fetuin samples and their labeling intensities (indicated as − and +) are shown in FIG. 6. The glycosidases included C. perfringen neuraminidase, F. meningosepticum PNGase F that is selectively active on various N-glycans, and O-glycosidase that is specific for core-1 (Galβ1-3GalNAc-O-S/T) and core-3 (GlcNAcβ1-3GalNAc-O-S/T) O-linked disaccharides.

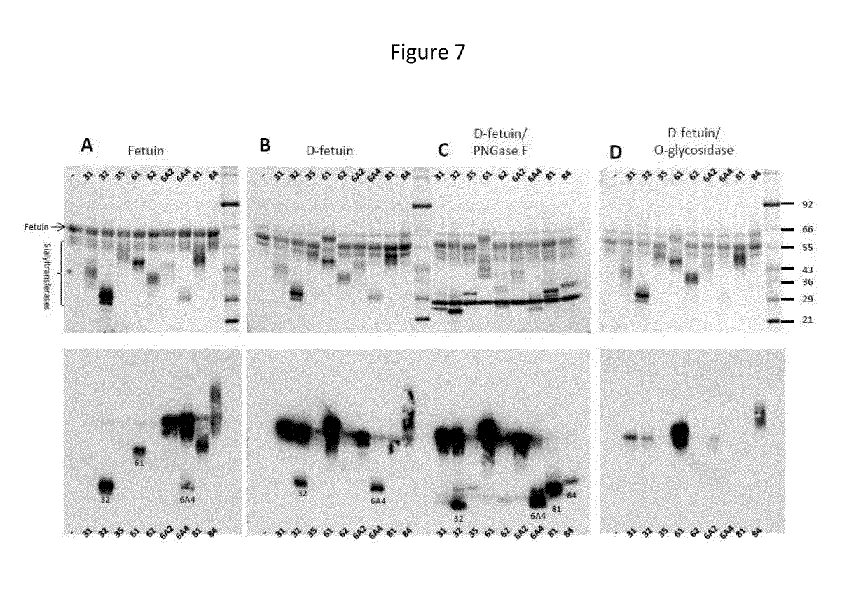

Untreated or glycosidase treated fetuin samples were tagged with azido-sialic acid using the nine sialyltransferases and then conjugated to a biotin moiety through click chemistry reactions. The samples were separated on SDS-PGAE and then blotted to nitrocellulose membrane for detection with streptavidin-conjugated horse radish peroxidase (strep-HRP) using enhanced chemiluminescence as substrate. These results are shown in FIG. 7.

### Example 1

### Probing with β-Gal α-2-3-sialyltransferases

Fetal bovine fetuin samples were first probed with three α-2-3-sialyltransferases, ST3Gal1, ST3Gal2, and ST3Gal5. ST3Gal1 and ST3Gal2 transfer sialic acid to the disaccharide moiety of Galβ1-3GalNAc of glycoproteins and glycolipids such as core-1 O-glycan. ST3Gal5 is a sialyltransferase that synthesizes ganglioside GM3. All three enzymes failed to label untreated fetuin (as shown in part A of FIG. 7), but desialylated fetuin (d-fetuin) was strongly labeled by ST3Gal1 and ST3Gal2 (as shown in part B of FIG. 7), suggesting that substrate glycans are fully sialylated. Furthermore, ST3Gal1 and ST3Gal2 labeling on d-fetuin was resistant to native PNGase F digestion (as shown in part C of FIG. 7) but sensitive to O-glycosidase treatment (as shown in part D of FIG. 7), suggesting that the labeling is on core-1 or core-3 O-glycan. Considering that core-3 O-glycan is not a substrate for ST3Gal1 and ST3Gal2, the labeling must be on core 1 O-glycan. As expected, ST3Gal5 didn't have obvious labeling in all cases. All together, these results indicated the presence of fully sialylated core-1 O-glycan on fetal bovine fetuin.

Interestingly, ST3Gal2 also showed self-labeling that was down-shifted by PNGase F and abolished by O-glycosidase, indicating the presence of core-1 O-glycan on the enzyme itself. Since that ST3Gal2 was expressed in CHO cells, these results also suggest that sialylation on recombinant proteins by CHO cells may not be completed.

### Example 2

### Probing with β-Gal α-2-6-sialyltransferases

The samples were also probed with two α-2-6-sialyltransferases, ST6Gal1 and ST6Gal2. ST6Gal1 catalyzes the formation of NeuAca2-6Galβ1-4GlcNAc sequence on N-linked oligosaccharides of glycoproteins. ST6Gal2 catalyzes the formation of the same structure but exhibits relatively low or no activities toward glycoproteins and glycolipids ST6Gal1 didn't labeling on fetuin but strongly labeled d-fetuin, suggesting that the N-glycans on fetuin were also fully sialylated (as shown in parts A and B of FIG. 7). In contrast, ST6Gal2 had no labeling in all cases. The labeling by ST6Gal1 on d-fetuin was not sensitive to the treatment of O-glycosidase (as shown in part D of FIG. 7), suggesting that the labeling was unlikely on O-glycan. However, the labeling was also not sensitive to native PNGase F digestion either (as shown in part C of FIG. 7). More interestingly, re-sialylation by ST6Gal1 up shifted the band of d-fetuin from about 56-57 kDa to about 60-61 kDa (as shown in part B of FIG. 7), suggesting that there are about 9 to 15 acceptor sites for ST6Gal1 on each d-fetuin molecule.

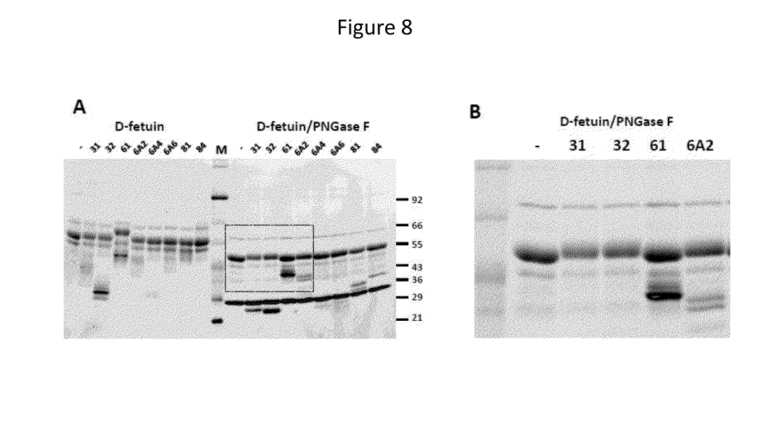

To test whether sialylation by ST6Gal1 only occurs on N-glycans, various resialylated d-fetuin samples along with d-fetuin itself were treated with PNGase F under denaturing condition (as shown in FIG. 8). This treatment down shifted ST6Gal1 resialylated samples and d-fetuin to exactly the same position (for a more clear view, see part B of FIG. 8), suggesting that ST6Gal1 labeling is indeed on N-glycans. In contrast, after the same treatment, ST3Gal1 and ST3Gal2 resialylated d-fetuin migrated slightly slower than that of d-fetuin, indicating that sialylation by these two enzymes are on glycans other than N-glycans, which further confirms the specificity of these enzymes for 0-glycans.

### Example 3

### Probing with GalNAc α-2-6-sialyltransferases

There are six GalNAc specific α-2,6-sialyltransferases. In this study, fetal bovine fetuin samples were probed with ST6GalNAc2 and ST6GalNAc4. ST6GalNAc 2 sialylates core-1 O-glycan (Galβ1-3GalNAc-O-Ser/Thr), sialyl core-1 O-glycan (Siaα2-3Galβ1-3GalNAc-O-Ser/Thr) [30, 31] and Tn antigen (GalNAc-O-Thr/Ser). ST6GalNAc4 is known to be strict on utilizing sialyl core-1 O-glycan as substrate and generates disialylated tetrasaccharide Siaα2-3Galβ1-3(Siaα2-6)GalNAc. In contrast to β-Gal α-2-3 and α-2-6 sialyltransferases, ST6GalNAc2 and ST6GalNAc4 directly labeled fetal bovine fetuin (as shown in part A of FIG. 7), confirming an earlier finding of these two enzymes regarding their substrate specificities. ST6GalNAc2 labeling was not abolished by prior neuraminidase treatment (as shown in part A of FIG. 8) and afterwards native PNGase F treatment (as shown in part C of FIG. 7), but was sensitive to prior O-glycosidase treatment (as shown in part D of FIG. 7). Based on the substrate specificities of these enzymes, the major glycan labeled by ST6GalNAc2 is likely to be either core-1 or sialyl core-1 O-glycan. It is also possible that a minor amount of Tn antigen or STn antigen was labeled.

Labeling by ST6GalNAc4 confirms the presence of sialyl core-1 glycans on fetuin, which again is consistent with the results of ST3Gal1 and ST3Gal2 probing. In sharp contrast to ST6GalNAc2, ST6GalNAc4 labeling was sensitive to prior neuraminidase treatment (as shown in part B of FIG. 7), confirming that the sialylation of the Gal on core-1 O-glycan is critical for the recognition by ST6GalNAc4.

### Example 4

### Probing with α-2-8-sialyltransferases

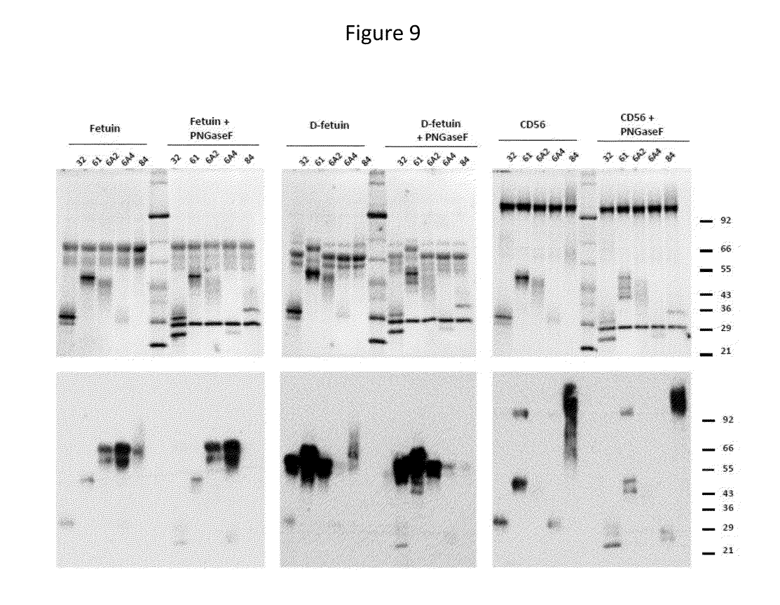

The fetuin samples were also probed with two α-2-8-sialyltransferases, ST8Sia1 and ST8Sia4. ST8Sia1 catalyzes the transfer of sialic acid from CMP-sialic acid to GM3 (NeuNAca2-3Galβ1-4GlcCer) to produce GD3 (NeuNAca2-8NeuNAca2-3Galβ1-4GlcCer) and GT3 (NeuNAca2-8NeuNAca2-8NeuNAca2-3Galβ1-4GlcCer) in a successive manner. ST8Sia4 is known to generate polysialic acid (PSA) on neural cell adhesion molecules during embryonic development and catalyzes both the addition of the first α-2,8-linked sialic acid to α-2,3-linked sialic acid and the polycondensation of all α-2,8-linked sialic acids, yielding PSA. Both enzymes showed some labeling (as shown in FIG. 7), however, the band patterns were different from labeling by other sialyltransferases. The ST8Sia1 labeled band moved faster and ST8Sia4 labeled band moved slower than the labeling on fetuin by other enzymes. The ST8Sia1 labeled band was downshifted by native PNGase F treatment to about 36 kDa (as shown in part C of FIG. 7), and abolished by O-glycosidase, suggesting that the labeling is on the O-glycan of the enzyme itself. The ST8Sia4 labeled band was downshifted by PNGase F treatment to about 37 kDa (as shown in part C of FIG. 7) and resistant to O-glycosidase treatment, suggesting that labeling is mainly on its own N-glycans. The predicted molecular weights for ST8Sia1 and ST8Sia4 are around 36 kDa and 37 kDa respectively. These conclusions were further supported by labeling with a known substrate for ST8Sia4, CD56 (as shown in FIG. 9).

### Example 5

### Probing with GlcNAc Transferases

To confirm the results obtained through sialyltransferase probing, fetal bovine fetuin samples were also probed with GlcNAc transferases, including recombinant human MGAT1, GCNT1, and B3GNT6. MGAT1 transfers a GlcNAc to the α-3 linked mannose of the trimannosyl core of N-linked oligosaccharides and initiates the formation of complex and hybrid N-linked carbohydrates; therefore MGAT1 probing reveals the presence of high mannose or hybrid glycans that contain unmodified α-3 linked mannose. By adding a GlcNAc to the GalNAc, GCNT1 converts the core-1 O-glycan to core 2 O-glycan; therefore probing with GCNT1 reveals the presence of core-1 O-glycan. B3GNT6 transfers a GlcNAc to Tn antigen and forms core-3 structure (GlcNAcβ1-3GalNAc-O-T/S); therefore B3GNT6 probing reveals the presence of Tn antigen.

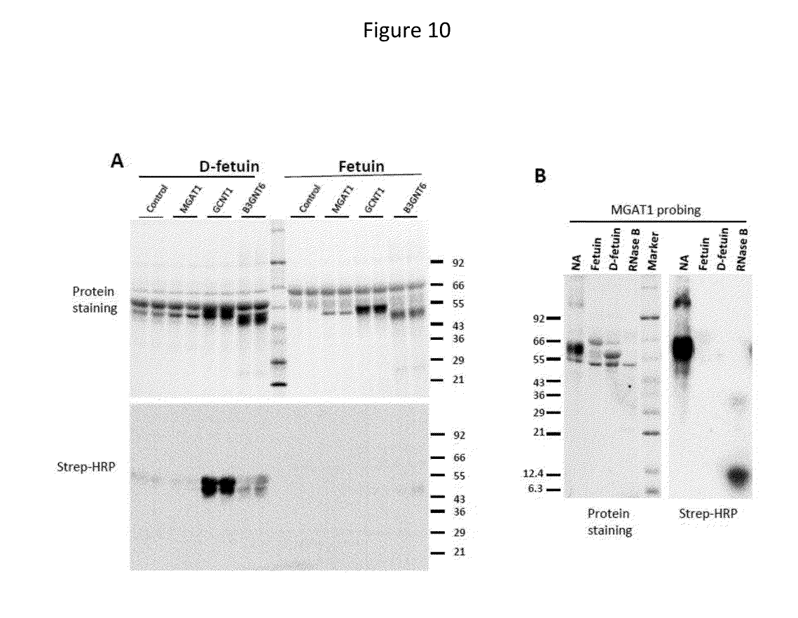

When fetuin and d-fetuin were probed with MGAT1, no labeling was observed (as shown in part A of FIG. 10), suggesting that there are no high-mannose or hybrid N-glycans that contain unmodified α-3 linked mannose. As controls, 1918 H1N1 neuraminidase expressed in insect cells and bovine RNase B that are known for containing high mannose N-glycan structures were strongly labeled with MGAT1 (as shown in part B of FIG. 10). When probed with GCNT1, d-fetuin but not fetuin was strongly labeled, again suggesting the presence of sialyl core-1 O-glycan on fetuin and supporting the notion that sialylation on core-1 prevents the action of GCNT1. B3GNT6 had some weak labeling on d-fetuin but not fetuin, suggesting the presence of minute amount of STn antigen on fetuin.

### Discussion

In examples 1-5, sialoglycans were probed on fetal bovine fetuin. Fetal bovine fetuin contains three N-linked complex glycans and three O-linked glycans with the N-glycans accounting for 79% of the total glycans. The known O-glycans on the protein include a disialylated core-1 O-glycan (Siaα2-3Galβ1-3(Siaα2-6)GalNAc), a monosialylated core-1 O-glycan (Siaα2-3Galβ1-3GalNAc) and a disialylated hexasaccharide (Siaα2-3Galβ1-3 [Siaα2-3Gal β1-4GlcNAc β1-6]GalNAc). The probing showed that non-reducing end Gal s of both glycans are fully sialylated, while the O-GalNAc s are not. The probing also showed that the protein lacks high mannose N-glycans and epitopes for polysialylation. The probing also demonstrated the presence of sialyl core-1 O-glycans using not only sialyltransferases but also GlcNAc transferases.

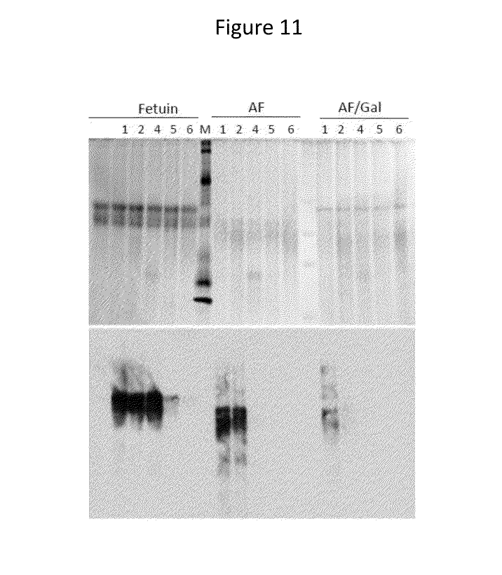

The results also suggest the presence of a minute amount of STn antigen on fetal bovine fetuin. STn is mainly synthesized by ST6GalNAc1; however, ST6GalNAc1 was not used for specific detection of Tn antigen, because the enzyme is also active on sialyl core-1 and core-1 O-glycans (as shown in FIG. 11). Instead, B3GNT6 was used for the detection because of its high specificity for Tn antigen.

While almost complete sialylation on non-reducing end Gal s was found on fetal bovine fetuin, incomplete sialylation was found on some of the sialyltransferases themselves. One possible cause for this difference on sialylation is that proteins in circulation with exposed non-reducing end Gal s are recognized and cleared away by asialoglycoprotein receptor in the liver, which may not be happening during cell culture. Another possible cause is that some of the host cells for recombinant protein expression may have a lower level of or a lack of certain sialyltransferases, for example, CHO cell lacks ST6Gal1.

Various examples have been described. These and other examples are within the scope of the following claims.

1. An in vitro method of determining presence or absence of a target carbohydrate on a target glycoprotein, comprising:
providing a sample containing a target glycoprotein;
treating the sample with a glycosidase, wherein the glycosidase removes a target carbohydrate if present on the target glycoprotein, thereby creating a vacant glycosylation acceptor site;
treating the sample with a glycosyltransferase to incorporate a replacement carbohydrate into the vacant glycosylation acceptor site if present, wherein the replacement carbohydrate includes a click chemistry moiety;
adding a label to the sample, wherein the label includes a click chemistry moiety that reacts to the click chemistry moiety of the replacement carbohydrate such that that the label attaches to the replacement carbohydrate;
separating the sample using a separation method;
determining presence or absence of label attached to the replacement carbohydrate in the separated sample; and
correlating presence of the label to presence of the target carbohydrate or correlating absence of the label to absence of the target carbohydrate.

2. The method of claim 1 further comprising correlating presence of the target carbohydrate to presence of a glycan epitope or correlating absence of the target carbohydrate to absence of the glycan epitope.

3. The method of claim 1 wherein the separation method is an electrophoresis method.

4. The method of claim 1 wherein the replacement carbohydrate includes a click chemistry moiety selected from one of an azido group or an alkyne group and the label includes a click chemistry moiety selected from the other of the azido group or the alkyne group.

5. The method of claim 1 wherein the label includes a label selected from a biotin molecule, a fluorescent molecule or a luminescent molecule.

6. The method of claim 1 wherein the determining presence or absence of the label in the separated sample comprises subjecting the separated sample to a blotting method to visualize the label.

7. The method of claim 1 wherein the target carbohydrate is a target carbohydrate selected from the group consisting of sialic acid, fucose, GlcNAc, GalNAc, galactose, mannose and xylose.

8. The method of claim 1 wherein the glycosidase is a glycosidase selected from the group consisting of sialidase, fucosidase, hexosaminidase and galactosidase or combinations thereof.

9. The method of claim 1 wherein the glycosyltransferase is a glycosyltransferase selected from the group consisting of sialyltransferases, fucosyltransferases, GlcNAc transferases, GalNAc transferases, galactosyltransferases, glucosyltransferases, xylosyltransferases, mannosyltransferases and combinations thereof.

10. An in vitro method of determining presence or absence of a target carbohydrate on a target glycoprotein, comprising:
providing a sample containing a target glycoprotein;
aliquoting the sample into a first aliquot and a second aliquot;
treating the first aliquot with a glycosidase and leaving the second aliquot untreated, wherein the glycosidase removes a target carbohydrate if present to create a vacant glycosylation acceptor site on the target glycoprotein;
treating each the first aliquot and the second aliquot with a glycosyltransferase, wherein the glycosyltransferase incorporates a replacement carbohydrate into the vacant glycosylation acceptor site if present, wherein the replacement carbohydrate includes a click chemistry moiety;
adding a label to each the first aliquot and the second aliquot, wherein the label includes a click chemistry moiety that reacts to the click chemistry moiety of the replacement carbohydrate such that that the label attaches to the replacement carbohydrate;
performing a separation method on each the first aliquot and the second aliquot;
determining presence or absence of label attached to the replacement carbohydrate in each the first aliquot and the second aliquot; and
correlating presence or absence of label in the first aliquot and the second aliquot to presence of a target carbohydrate.

11. The method of claim 10 further comprising correlating presence of the target carbohydrate to presence of a glycan epitope or correlating absence of the target carbohydrate to absence of the glycan epitope.

12. The method of claim 10 wherein the separation method is an electrophoresis method.

13. The method of claim 10 wherein the replacement carbohydrate includes a click chemistry moiety selected from one of an azido group or an alkyne group and the label includes a click chemistry moiety selected from the other of the azido group or the alkyne group.

14. The method of claim 10 wherein the label includes a label selected from a biotin molecule, a fluorescent molecule or a luminescent molecule.

15. The method of claim 10 wherein the determining presence or absence of label attached to the target carbohydrate in each the first aliquot and the second aliquot comprises subjecting the separated each the first aliquot and the second aliquot to a blotting method to visualize the label.

16. The method of claim 10 wherein the target carbohydrate is a target carbohydrate selected from the group consisting of sialic acid, fucose, GlcNAc, GalNAc, galactose, mannose and xylose.

17. The method of claim 10 wherein the glycosidase is a glycosidase selected from the group consisting of sialidase, fucosidase, hexosaminidase and galactosidase or combinations thereof.

18. The method of claim 10 wherein the glycosyltransferase is a glycosyltransferase selected from the group consisting of sialyltransferases, fucosyltransferases, GlcNAc transferases, GalNAc transferases, galactosyltransferases, glucosyltransferases, xylosyltransferases, mannosyltransferases and combinations thereof.

19. An in vitro method of determining presence or absence of target carbohydrates on a target glycoprotein, comprising:
providing a sample containing a target glycoprotein;
aliquoting the sample into a first aliquot and a second aliquot;
treating the first aliquot with a glycosidase and leaving the second aliquot untreated, wherein the glycosidase removes a target carbohydrate if present to create a vacant glycosylation acceptor site on the target glycoprotein;
further aliquoting the first aliquot into a first subgroup and the second aliquot into a second subgroup;
treating each aliquot in the first subgroup and the second subgroup with a different glycosyltransferase, wherein each glycosyltransferase includes a single glycosyltransferase or a plurality of glycosyltransferases, wherein each glycosyltransferase incorporates a target carbohydrate into the vacant glycosylation acceptor site if present, wherein the target carbohydrate includes a click chemistry moiety;
adding a label to each aliquot in the first subgroup and the second subgroup, wherein the label includes a click chemistry moiety that reacts to the click chemistry moiety of the target carbohydrate such that that the label attaches to the target carbohydrate;
performing a separation method on each aliquot in the first subgroup and the second subgroup;
determining presence or absence of the label in each aliquot in the first subgroup and the second subgroup; and
correlating presence or absence of the label to presence or absence of target carbohydrates.

20. The method of claim 19 further comprising correlating presence of a target carbohydrate in the target carbohydrates to presence of a glycan epitope.

21. An in vitro method of determining presence or absence of target carbohydrates on a target glycoprotein, comprising:
providing a sample containing a target glycoprotein;
aliquoting the sample into a plurality of aliquots;
leaving one aliquot in the plurality of aliquots untreated and treating remaining aliquots in the plurality of aliquots with a different glycosidase, wherein each glycosidase includes a single glycosidase or a plurality of glycosidases, wherein each glycosidase removes a target carbohydrate if present to create a vacant glycosylation acceptor site on the target glycoprotein;
further aliquoting each aliquot in the plurality of aliquots into a subgroup;
treating each aliquot in each subgroup with a different glycosyltransferase, wherein the glycosyltransferase includes a single glycosyltransferase or a plurality of glycosyltransferases, wherein each glycosyltransferase incorporates a target carbohydrate into the vacant glycosylation acceptor site if present, wherein the target carbohydrate includes a click chemistry moiety;
adding a label to each aliquot in each subgroup, wherein the label includes a click chemistry moiety that reacts to the click chemistry moiety of the target carbohydrate such that that the label attaches to the target carbohydrate;
performing a separation method on each aliquot in each subgroup;
determining presence or absence of the label in each aliquot in each subgroup; and
correlating presence or absence of the label to presence or absence of target carbohydrates.

22. The method of claim 21 further comprising correlating presence of a target carbohydrate in the target carbohydrates to presence of a glycan epitope.
